(19) United States
(12) Patent Application Publication
(10) Pub. No.: US 2023/0201025 A1
(43) Pub. Date: Jun. 29, 2023

(54) PROCESS FOR FABRICATING A DENTAL APPLIANCE AND ACCOMPANYING USE CASES

(71) Applicant: Bottmedical AG, Basel (CH)

(72) Inventors: Tino TÖPPER, Freiburg (DE); Bekim OSMANI, Basel (CH); Elise DARD, Basel (CH)

(73) Assignee: Bottmedical AG, Basel (CH)

(21) Appl. No.: 18/021,019

(22) PCT Filed: Aug. 6, 2021
(86) PCT No.: PCT/EP2021/072046

§ 371 Date: Feb. 13, 2023

(30) Foreign Application Priority Data
Aug. 14, 2020 (EP) 20191189.8

(51) Int. Cl. A61F 5/56; A61C 7/08; A61K 9/00

(52) U.S. Cl. CPC A61F 5/566; A61C 7/08; A61K 9/0063

(57) ABSTRACT

For improving the functionality and bio-compatibility of dental appliances (2), a novel process is described for making a thermoplastic functional foil (1), from which such dental appliances (2) may be obtained by thermoforming the functional foil (1). A carrier liquid (9) containing an organic and preferably bio-based polymer (6) is enriched with an agent (7) and applied onto a solid thermoplastic core foil (13). After evaporating of a solvent contained in the carrier liquid (9), a uniform and highly homogenous functional coating (5) is obtained on the core foil (13). After thermoforming of the foil (1), the dental appliance (2) thus features an outer protective coating (5) offering enhanced functionality. Moreover, it is possible to reload the agent (7) into the coating (5) of the appliance (2) using a reload-liquid (23).

### CROSS-REFERENCE TO RELATED APPLICATIONS

This application is the U.S. National Phase of PCT Appln. No. PCT/EP2021/072046, filed Aug. 6, 2021, which claims priority to European Patent Application No. 20191189.8, filed Aug. 14, 2020, the entire disclosures of which are incorporated by reference herein.

### TECHNICAL FIELD

The present disclosure relates to a process for making a thermoplastic functional foil comprising a core of thermoplastic material, in particular a core made from a thermoplastic polymer, and a thermoplastic coating at least partially covering the core.

This foil is particularly suited for intra-oral use as a dental appliance to be worn inside the oral cavity on the teeth, since such an appliance may be easily formed from such a foil. Hence the disclosure also concerns a dental appliance fabricated, in particular formed, most preferably thermoformed, from such a functional foil. The dental appliance may be used as an orthodontic appliance or a protective appliance for protection of teeth and/or gingiva of the user of the appliance.

Finally the disclosure concerns a novel non-therapeutic use-case, in which a dental appliance, as described before, is worn by a user for protecting his teeth and/or gingiva, in particular from cigarette smoke and/or from resulting gingivitis and/or parodontitis; moreover, by wearing the appliance on the teeth, the formation of plaque can be reduced and thus the risk to develop caries.

### BACKGROUND

In the past, intraoral orthodontic devices have been used in orthodontic therapy for realigning teeth. In this specific application case, the devices are typically designed as a dental splint, which is applied to teeth over several hours. The dental splint exerts forces on the individual teeth of the patient, which results in a realignment of the teeth in directions of desired teeth positions. For this purpose, typically a number of dental splints, which vary slightly from each other in shape, are worn by the patient consecutively to realign the teeth step-wise.

Further known applications of such intraoral orthodontic devices, in particular dental splints, are bleaching of teeth using bleaching pastes which are applied to the teeth by the device, or use of such devices for treating snoring overnight.

Intraoral appliances such as orthodontic devices are also used for avoiding clenching and grinding. Chronic teeth clenching and teeth grinding can cause overuse of the muscles controlling the lower jaw, leading to pain from those muscles. The load on the joint itself can also cause changes inside the joint, leading to pain and limited opening of the mouth.

In all of these applications, providing a high wearing comfort of the device is of central importance for a high acceptance of the treatment by the patient and hence overall success of the therapy. But also when a dental appliance is worn purely for protection of teeth, a high wearing comfort is an important quality.

### SUMMARY

In this context, the invention aims at further enhancing the functionality and biocompatibility of such dental appliances and—in relation to this fundamental aim—at providing an efficient method for fabricating such devices.

In accordance with the present invention, a process is provided, which solves the afore-mentioned problem. In particular, the invention proposes a process for making a thermoplastic functional foil as described at the beginning featuring a thermoplastic core and a thermoplastic coating at least partially, but preferably fully, covering the core. This process comprises the following steps: providing a carrier liquid comprising an organic polymer, which may preferably be a thermoplastic polymer; and applying the carrier liquid as the coating onto the core.

Particularly suitable materials for the core are polyethylene terephthalate (PET), in particular PETG, polycarbonate (PC), and polyurethane (PU). These materials may also be used in recycled form (rPET, rPU, rPET, rPETG).

In other words, the invention suggest to apply the coating in the form of liquid coating onto a solid core, which may have the form of a core foil, in particular onto both sides of such a core foil, and to solidify the liquid coating afterwards on the core to form a solid thermoplastic coating covering the core.

Through this process, a highly uniform and homogenous coating of the core can be achieved, and both the core and the coating can be thermoformed together in a later process step, as will be explained in greater detail below. Another great advantage of this liquid coating approach is that the coating can provide new functionalities to the foil and dental appliances formed from the foil. In particular, as will be explained in greater detail below, an agent may be mixed into the carrier liquid forming the coating prior to solidifying the coating on the core.

The coating may be formed, alternatively, using a hot melt extrusion process and providing the carrier liquid as a carrier melt. Such a carrier melt may also be applied onto a solid core to form a functional coating.

As will become clear below, the coating may form a thermoplastic cap layer of a dental appliance fabricated from the foil. For particular applications, for example when antimicrobial protection is to be provided, it can be beneficial if the cap layer fully covers the core of the device, and hence the thermoplastic coating may fully cover the core of the functional foil.

According to the invention, there exist further advantageous examples solving the aforementioned problems, which are described in the following:

For example, one example of the process suggests to add an agent to the carrier liquid prior to forming the coating. Preferably, the agent may be added to the carrier liquid in the form of an agent liquid, as many agents can be obtained already in liquid form. Such an agent may be in particular an agent that is releasable from the coating in aqueous environments. This has the advantage that such an agent can be released from the coating into an oral cavity of a user that is wearing a dental appliance formed from the functional foil.

Another highly preferred example of the process suggests that the agent is derived from a bio-based material. In other words, the agent may be derived from a renewable biological resource, in particular a material of non-fossil origin produced by a living organism. The term “bio-based material” may be understood here in the sense that a bio-based material may comprise a non-fossil carbon content containing a detectable portion of the carbon isotope ¹⁴C. Preferably, the non-fossil carbon content may constitute at least 20%, preferably at least 35%, most preferably more than 50% of the bio-based material. In other words, about 80% to 65% but preferably much less of the bio-based material can be based on other organic material, in particular polymers, derived from fossil sources such as crude oil.

As will be explained later, such a bio-based material may also be used as the material for the coating and/or such a bio-based material may fully cover a core of a dental appliance fabricated from said functional foil, with the core not being made from a bio-based material.

¹⁴C is a radioactive isotope produced in the earth's atmosphere at a concentration of about 1.25.10⁻¹²=1250 ppq (ppq=part per quadrillion=10⁻¹⁵) and incorporated into plants by photosynthesis. If a plant dies, it stops exchanging carbon with its environment, and thereafter the amount of ¹⁴C it contains begins to decrease as the ¹⁴C undergoes radioactive decay. The older a sample containing such biomaterial is, the less ¹⁴C remains to be detected. Hence by detecting the presence of ¹⁴C, material containing non-fossil carbon content can be differentiated from material derived from fossil sources. The presence of the ¹⁴C in a material is thus a direct proof that the material is obtained, at least in part, from non-fossil renewable biological sources. This is because organic polymers derived from fossil oil for example do not show any ¹⁴C any more, since the half-time of radioactive ¹⁴C is less than 6.000 years.

“Detectable portion” may be understood here in that the fraction of the ¹⁴C among all C-atoms of the non-fossil carbon content of the bio-based material may be at least 1 ppq, preferably at least 10 ppq or even at least 100 ppq.

The bio-based material may be further characterized in that it comprises a bio-based content of at least 40%, preferably of at least 50%, and wherein said bio-based content includes the non-fossil carbon content containing ¹⁴C described above as well as nitrogen, oxygen and hydrogen bound to the non-fossil carbon content.

In other words, at least part of the coating may be based on one or more bio-based materials. Preferably, however, the coating may be fabricated exclusively from bio-based materials.

Most preferably, the agent may be derived from an essential oil. Such an oil may be extracted directly from non-fossil plants. Such features ensure that the bio-based agent is not harmful to the body of the user of the dental appliance. Moreover, with such an approach, a myriad of different functionalities such as adding flavor or antimicrobial protection may be achieved by relying on natural products that do not lead to harmful contaminations inside the oral cavity.

According to another preferred example, the agent may comprise at least one of the following substances: an essential oil, an extract of an essential oil, or a derivative from the chemical family of phenols, monoterpenols, aldehydes, ketones, oxide terpenes, ethers, monoterpenes, lactone, phthalides, terpenic aldehydes, sestequiterpen alcohols, sestequiterpen, terpene esters, coumarins, in particular a substance such as cinnamaldehyde, lime, thymol, eugenol, linalool, carvacrol, nutmeg, pimenta berry, rosemary, petitgrain, coffee, anise, spearmint.

Further extracts/derivatives obtained from bio-based materials, in particular through extraction from a natural essential oil, that may be used as an agent to be embedded in the coating, are +α, β-pinene, myristicin, methyl eugenol, menthol, terpinene, p-cymene, linalool, myrcene, pinene, β-ocimene, geranial, sabinene, neral, citronellol, linolenic acid, oleic, stearic, himachalene, bisabolene, trans-cinnamaldehyde, methyl salicylate, eucalyptol, trans-anethole, (+)-limonene, (−)-limonene or L-carvone.

A particular advantage of using lime and cinnamaldehyde as agents is that they are not only highly effective antimicrobial agents but also provide a softening and plasticizing effect to the thermoplastic coating.

To further enhance the safety and compliance of dental appliances formed from the functional foil, it is highly preferable, if the organic polymer contained in the carrier liquid is a bio-based material or at least derived from a bio-based material. In other words, the organic polymer (forming the coating) may be derived from a renewable biological resource, in particular a material of non-fossil origin produced by a living organism.

A particular suitable choice for the organic polymer (forming the coating) is a cellulose-based material, in particular a cellulose-based composite. This is because cellulose is a bio-based material that can render the surface of the dental appliance soft, after swelling in water; in addition, cellulose offers favorable chemical interactions with the mentioned agents to be loaded or reloaded into the CAP layer comprising cellulose based composites.

Suitable materials for fabricating the core of the foil are cellulose acetate, cellulose acetate butyrate, PLA, chitosan or a polyester, a co-polyester, polycarbonate, polyurethane, polypropylene, polyethylene, polypropylene and polyethylene copolymer, acrylic, cyclic block copolymer, polyetheretherketone, polyamide, polyethylene terephthalate, polybutylene terephthalate, polyetherimide, polyethersulfone, polytrimethylene terephthalate or a combination thereof (e.g., a blend of at least two of the listed hard polymeric materials). In some embodiments, the core of the device can include polymeric materials, such as polycarbonate, a co-polyester, a polyester, and polyurethane. Moreover, the core can also be composed of multiple layers, e.g., two or more polymer layers.

Preferably, however, the core should mainly consist of a thermoplastic polymer (to render the foil thermoformable).

Another highly preferred choice is to use cellulose acetate butyrate (CAB) as the organic polymer (i.e. for the coating). In this case, it is highly preferable for good mechanical properties of the thermoplastic functional foil (and dental appliances formed from the foil), if a butyryl content of the CAB is between 15 to 60% by weight.

By providing such features, it can be achieved that the agent embedded into the coating is releasable from the coating, in particular in aqueous environments, for example in the oral cavity. Moreover, it can be achieved by adequate choice of the agent, for example by using cinnamaldehyde as the agent, that the coating provides antimicrobial protection (through the release of an antimicrobial agent). Hence, the agent may be an antimicrobial agent.

To improve the malleability and adaptiveness of the foil during thermoforming, it is of advantage if a glass transition temperature T_{g,coating} of the coating, and/or in particular a glass transition temperature of the organic polymer T_{g,polymer}, is/are in the range of 80-165° C. This is particularly true when using CAB as the organic polymer.

A suitable corresponding melting range of the coating, and in particular said organic polymer, may be from 120-200° C.

Another important factor for achieving satisfying results when thermoforming the functional foil is the number average molecular weight of the organic polymer used for the coating. For optimum results, the number average molecular weight may be more than 12.000 g/mol, preferably more than 20.000 g/mol, and most preferably more than 30.000 g/mol. Such numbers are particularly suited if CAB is chosen as the organic polymer.

Another highly advantageous example of the process introduced at the beginning proposes that the carrier liquid is a carrier solution, i.e. the carrier liquid may be a solution of a solvent and the organic polymer. In this case, it if preferable if the carrier solution is obtained by dissolving the organic polymer in a solvent. This has the advantage that the coating may be solidified on the core by evaporating the solvent from the coating. This approach is of advantage because the solvent can be used to modify the surface of the core, as will be explained later. As already said, the core may actually be a core foil and hence, the carrier solution may be applied to both sides of the core foil uniformly.

For achieving a uniform coating thickness, the coating can be formed by a physical coating process such as spin coating or blade-based coating, or by spray coating or by roll-to-roll coating. Of course there is a myriad of different processes are available for performing roll-to-roll coating (e.g. air knife coating, gap coating, immersion dip coating, roller coating, etc.) or spray coating (spray painting, thermal spraying, plasma spraying etc.), as examples.

As already mentioned, the solvent of the carrier solution can be used to modify the surface of the core. In fact, the solvent can soften the core's surface by reducing the interaction of molecular chains of the material of the core. As a result, after treating the core with the carrier solution, the core and the coating (or to be more precise, the organic polymer of the coating) can interlock on a nanometer scale, resulting in improved adhesion of the coating on the core. After application of the coating in liquid form onto the core and interaction of the solvent with the core, the solvent may be simply evaporated thus forming a final solid coating.

Such as softening of the core can be achieved, for example, when using acetone as the solvent and PETG as the core material, but there exist other combinations of core materials and suitable solvents, which achieve the same softening effect, as is readily apparent in the literature. The invention suggests to use this effect for enhancing the adhesion of the coating on the core of a functional foil intended for fabricating a dental appliance.

Another approach for improving the mechanical and/or chemical interaction of the coating and the core is to safeguard that a glass transition temperature T_{g,core} of the core and a glass transition temperature T_{g,coating} of the coating differ by less than 80° C., preferably by less than 60° C. Such a material choice will result in enough mobility of the molecular chains of both core and coating such that efficient thermal interlocking can be achieved through thermal fusion (sometimes referred to as “thermal welding”) between core and coating during the final thermoforming of the functional foil.

Moreover, in such a case, it can be of advantage, if the glass transition temperature of the coating T_{g,coating} is higher than the glass transition temperature of the core T_{g,core}. Such a material choice will result in a sufficient mechanical stability during thermoforming of the device, as the organic polymer can provide some stability to the foil. For example, PETG, which is a useful material for the core, can have a glass transition temperature of 80° C., and a melting temperature as high as 210° C. Such a core can be combined with a coating based on cellulose acetate butyrate (CAB), and such a CAB-coating may feature a glass transition temperature T_{g,coating} of 120° C., or above, but at the same time a relatively low melting temperature T_{m,coating} of 160° C.

Additionally, either the coating should reach its melting temperature T_{m,coating} before the core reaches its melting temperature T_{m,core} or the core should reach its melting temperature before the coating reaches its own. This may be achieved—in particular when considering thermoforming—by safeguarding that a melting temperature of the core T_{m,core} and a melting temperature of the coating T_{m,coating} differ by at least 20° C., preferable at least 40° C., most preferably at least 60° C. As a result, a compound material may be produced at the interface between core and coating during thermoforming of the functional foil, because when for example the coating reaches its melting temperature, the core may still be in the glass transition phase/temperature range (or vice versa). This compound material will thus comprise chains from the organic polymer of the thermoplastic coating and parts of the material used for the thermoplastic core.

Preferable for easy fabrication, however, will be a design, in which the melting temperature of the core T_{m,core} is at least 20° C., preferable at least 40° C., most preferably at least 60° C., higher than the melting temperature of the coating T_{m,coating}. In such a design, the coating will become molten while the core material is still solid or still in the glass transition stage. This is highly beneficial for obtaining a robust process control, when thermoforming the foil.

The solvent used for the carrier solution may comprise at least one of acetone, methyl acetate, ethyl acetate, methylethyl ketone, isopropyl acetate, butyl acetate, ethyl lactate, cyclohexane, diacetone alcohol, butyl lactate or suitable mixtures of these solvents.

Another variant of the process is defined in that the agent itself may be a liquid. In this case, the agent in liquid form (agent liquid) may be simply mixed with the carrier liquid.

Alternatively, the agent may be added to the carrier liquid by dissolving or emulsifying the agent first in an aqueous or oil-based solution and mixing this solution (agent liquid) with the carrier liquid. In such a case, it is preferable for a homogenous distribution of the agent within the final coating, if the solution containing the agent is mixed with the carrier liquid, for example by a stirrer, to form a homogenous carrier solution, prior to forming the coating.

Concerning the ratio between the agent and the organic polymer in the final coating, this ratio may be typically between 0.01/99.99 and 30/70 by weight, for example 2-3 g of agent and 7 g of organic polymer. For most applications, it will be preferable if the ratio between the agent and the organic polymer is between 0.1/99.9 and 20/80 by weight.

A particular suitable process can be obtained, if the core of the functional foil is made from Polyethylenterephthalat (PET). In particular, the core may be made at least partly from recycled PET. In other words, the thermoplastic material of the core may comprise recycled PET.

For improving the mechanical properties of the functional foil, in particular for reducing the tendency for crack formation, glykol modified Polyethylenterephthalat (PETG) may be chosen as the material for the core. In these cases, a highly suitable solvent for achieving the softening effect described above is acetone.

The process may be further modified in that a natural softening agent, preferably triacetin or polycaprolactone-triol (PCL-T), is added as a plasticizer to the carrier liquid prior to forming the coating. This may be done, preferably, at a volume ratio of less than 40%, preferably of less than 20%, most preferably of less than 10%.

Finally, the process explained so far may be continued by forming a dental appliance from the functional foil by thermoforming the foil. Importantly, this thermoforming may be done after application of the coating onto the core, as both the core and the coating may be thermoplastic. As a result, after thermoforming of the functional foil, the organic polymer may form a conformal functional coating, preferably fully covering the core of the foil, which then constitutes the core of the dental appliance. Moreover, an agent previously embedded in the coating (as described above) may then provide antimicrobial protection or regenerative functionality for teeth and gingiva or a flavor or simply a nice color to the dental appliance.

An important aspect with regard to the final thermoforming is the thermal stability of the agent. In order to safeguard the proper functionality of the final device, a decomposition temperature of the agent T_{d,agent} should be at least 10° C. above the melting temperature T_{m,coating} of the coating (the decomposition temperature T_{d,agent} may thus lie up to 60° C. above the glass transition temperature T_{g,coating} of the coating).

Following the concept outlined above, the afore-mentioned problem may thus be solved also by a dental appliance according to the invention. As described at the beginning, this dental appliance may be an orthodontic appliance or a protective appliance for protection of teeth and/or gingiva, and it may be formed, in particular thermoformed, from a functional foil fabricated with a process as described herein or as described by one of the claims directed towards such a process.

Alternatively, the dental appliance may be 3d-printed (directly) from a liquid precursor comprising an organic polymer (and optionally the agent). In this case, the dental appliance may be (re-)loaded with a releasable agent after fabrication using a reload-liquid, as will be explained in greater detail below.

In such a dental appliance, the agent described above with respect to the functional foil or an agent embedded into the dental appliance (preferably into a cap layer of the appliance) may be releasable from the dental appliance during intra-oral use. In addition or as an alternative, said agent may be reloadable into a cap layer of the dental appliance using a reload-liquid (to be described further below).

According to a particular approach intended for using the dental appliance purely for protection of teeth and gingiva, the dental appliance may consist entirely of the functional foil, which may have a low thickness of less than 400 μm, preferably of less than 300 μm, most preferably of less than 200 μm.

With respect to the properties of the final device, the invention thus proposes a dental appliance which may be described as featuring a core of thermoplastic material, a thermoplastic coating at least partially covering the core, and an agent embedded in the coating and releasable from the coating in aqueous environment. The core and/or coating and/or agent may be chosen and/or designed as has been described in detail above with respect to the fabrication process or as defined by features of one of the sub-claims directed towards this process.

Most preferably, the agent should be embedded in the coating with a surface density of at least 0.05 mg of the agent per cm² surface area of the coating, because this guarantees a release rate sufficient for achieving the desired functionality of the appliance when worn in the mouth. Such a surface density may be easily reached by using a reload-liquid as defined in one of the claims directed towards a use of such a reload-liquid and/or as described in detail further below.

When achieving such a surface density, a release rate of the agent from the dental appliance of at least 1 mg/m² surface area of the coating per hour over a period of 24 hours can be maintained, when the appliance is worn in the mouth; this is particularly true when using a cellulose-based coating and an agent such as Limonene, cinnamaldehyde, methyl Salicylate or trans-anethole. Such a release rate has been found to be adequate in particular when the agent is chosen to provide antimicrobial protection.

Following the above concepts, a novel use case of a dental appliance is suggested, to benefit from its increased functionality. In other words, a non-therapeutic use of a dental appliance is proposed for protecting teeth and/or gingiva of a user wearing the dental appliance on his/her teeth. This protection can be in particular from cigarette smoke and/or gingivitis and/or parodontitis and the dental appliance may be fabricated as previously described. The wearing of the protective dental appliance on the teeth can also provide protection from plaque formation and thus reduce the risk to develop caries. This is particularly true when using lime and cinnamaldehyde as an agent embedded in a coating of the dental appliance. The dental appliance may also be worn for cosmetic reasons, for example if used in combination with a bleaching agent, or as a night guard, for providing protection against plaque, bacteria and gingivitis and/or for providing good taste and smell.

Apart from the approach of fabricating such a dental appliance, the use case is characterized in that during use, the dental appliance covers both teeth and parts of the gingiva adjoining to the teeth, and in that the dental appliance features a functional coating offering antimicrobial protection. This protection may be provided, as has been explained above, in particular through a releasable antimicrobial agent and this agent may be embedded in the functional coating, in particular as described previously. Most preferably for this use case is a situation, in which the coating is formed from a bio-based material and/or the agent is also derived from a bio-based material. This further enhances the protection and safety when wearing the appliance.

Moreover, in such a use case, it is preferable if the dental appliance is formed with cavities matching the natural positions of the teeth of a user such that the appliance does not exert forces onto the teeth. This greatly improves the wearing comfort, which is important, if the user wants to profit from long-time protection by wearing the dental appliance over hours. For the same reason, it is regarded highly preferable, if the thermoplastic functional foil shows a maximum thickness of less than 700 μm, preferably of less than 500 μm, most preferably of less than 300 μm (after the final thermoforming process). This is because, in particular due to the liquid coating approach, the thickness of the functional coating may be less than 100 μm or even less than 50 μm, while the coating may be still providing sufficient antimicrobial protection, due to a high concentration of the agent inside the coating. In particular, such a dental appliance can be comfortably worn by a user while the user is smoking or sleeping.

In the use case of dental teeth alignment the embedded agent may also serve as a plasticizer, which reduces the brittleness of the thermoformed dental appliance and enables a long-term stable flexibility during a typical wearing period of two weeks (e.g. for each aligner in a series of aligners).

For providing greater flexibility in the choice of the agent or for replenishing the agent in the coating, after the dental appliance has been used multiple times, or for initially loading such an agent into a 3d-printed dental appliance, a use of a re-load liquid is proposed. This reload-liquid can load the agent into the dental appliance, in particular into the coating/cap layer described previously or into a 3d-printed body of the dental appliance.

The reloading may in particular be done initially in case no agent has been embedded in the appliance during its fabrication. On the other hand, the reload-liquid can also re-load an agent initially present in the coating but consumed through release from the appliance into the mouth again into the coating. Hence, the reload-liquid can be used both for loading or re-loading a dental appliance (which may be designed and/or fabricated as described previously) with a releasable agent.

The proposed use may be described in that the reload-liquid contains the agent and the dental appliance is immersed in the reload-liquid to load the agent into the dental appliance. The dental appliance may be fabricated by thermoforming or by direct 3D-printing, for example. In case of fabricating the dental appliance by 3d-printing, the agent may be embedded into a liquid precursor from which the appliance is printed.

For achieving sufficient loading, it is preferable if the reload-liquid contains a concentration of the agent of at least 0.2 g/l. In this case, a surface density of the agent embedded in the dental appliance of 0.05 mg/cm² can be achieved. This may result in a release rate from the dental appliance, in particular from said coating/said cap layer, of at least 1 mg/m² per hour, when the dental appliance is worn in the mouth during a typical wearing period of at least 24 hours.

In addition, it is also possible to use such a liquid for loading a releasable agent into a thermoplastic functional foil according to the invention, in particular prior to thermoforming.

The reload-liquid may be in the form of an aqueous solution or an oil-based liquid. It can also contain solvents such as ethanol. Such a solvent can reduce the surface tension and help in wetting the appliance with the reload liquid.

A particular convenient way is to obtain the reload-liquid from a liquid precursor comprising the agent (and preferably a surfactant to be described below), which may then be diluted to obtain the reload liquid.

An alternative approach is to obtain the reload-liquid by dissolving a solid tablet containing the agent in a liquid, preferably in water. Hence, it is proposed to use such a solid tablet for forming a reload-liquid and to use the load-liquid as described before. The tablet can therefore be soluble in water and the liquid used may be water.

The reload-liquid and/or the tablet just described may comprise a surfactant for homogenously distributing the agent within the reload-liquid. This is important for achieving a high load concentration of the agent per surface area of the coating.

One particularly suitable composition of such a tablet is a combination of an acidic material (e.g. citric acid), a base (e.g. sodium bicarbonate and citric acid), a fatty acid ester (e.g. Polyglyceryl-4 Laurate and/or Polyglyceryl-6 Caprate), a calcium derivative (e.g. calcium carbonate) and a sweetener (e.g. xylitol). To this composition, the agent or a combination of multiples agents, preferably and a surfactant (which may comprise a solubilizer and an emulsifier), may be added.

The surfactant may be derived from a renewable biological resource, in particular a material of non-fossil origin produced by a living organism. Preferred surfactants for this application are fatty acid esters, benzoic alcohols or glycerol-based surfactants. Further examples of surfactants particularly suitable for the application described herein are Polyglyceryl-4 Laurate or Polyglyceryl-6 Caprate. All of these substances can also be used in combination and have been found to be particularly suitable to be used with the agent cinnamaldehyde.

The concentration of surfactant and agent in the reload-liquid define the speed of loading the agent into the dental appliance. However, the ratio between surfactant and agent needs to be carefully balanced: On the one hand, the smaller the ratio of surfactant to agent, the faster the agent can diffuse from the reload-liquid into a cap layer of the dental appliance. In other words, the concentration of the surfactant should be as low as possible, since for excessive concentrations, the surfactant will capture the agent in the reload-liquid. On the other hand, the higher the concentration of surfactant and agent with respect to water in the reload liquid, the faster will be the (re-)loading.

A preferred ratio of the concentration cₛ of the surfactant and a concentration cₐ of the agent is cₛ/cₐ<100, most preferably cₛ/cₐ<10. Such ratios are particularly suited for the specific case of using Polyglyceryl-4 Laurate as an emulsifier and Polyglyceryl-6 Caprate as solubilizer (the surfactant thus comprises both of these components in this case). If the ratio cₛ/cₐ exceeds such values, there is a high likelihood that the agent will be captured (due to polar/non-polar interaction between single molecules) by the surfactant and not be embedded into the dental appliance.

Such parameters ensure, that a high absorption speed can be achieved, such that a significant amount of the agent (sufficient for producing a significant effect during wearing of the appliance) can be embedded into the coating of the dental appliance in less than an hour, or even less than 15 min (thus rendering the process of re-loading comfortable for the user). As a specific example, the amount of surfactant mₛ in the final reload-solution mₛₒₗ may be mₛ/mₛₒₗ>0.1 g/l.

Preferred examples of the present invention shall now be described in more detail, although the present invention is not limited to these examples: for those skilled in the art it is obvious that further examples of the present invention may be obtained by combining features of one or more of the patent claims with each other and/or with one or more features of an example described or illustrated herein.

### BRIEF DESCRIPTION OF THE DRAWINGS

With reference to the accompanying drawings, where features with corresponding technical function are referenced with same numerals even when these features differ in shape or design

FIG. 1 is a schematic overview of a fabrication process according to the invention,

FIG. 2 illustrates the definition of non-fossil carbon content and bio-based content used herein to describe bio-based materials,

FIG. 3 schematically illustrates a method for loading an agent into a dental appliance using a reload-liquid according to the invention,

FIG. 4 shows measurement data of concentrations of a particular agent loaded into different thermoplastic materials, and

FIG. 5 depicts further experimental data of calorimetric measurements.

### DETAILED DESCRIPTION

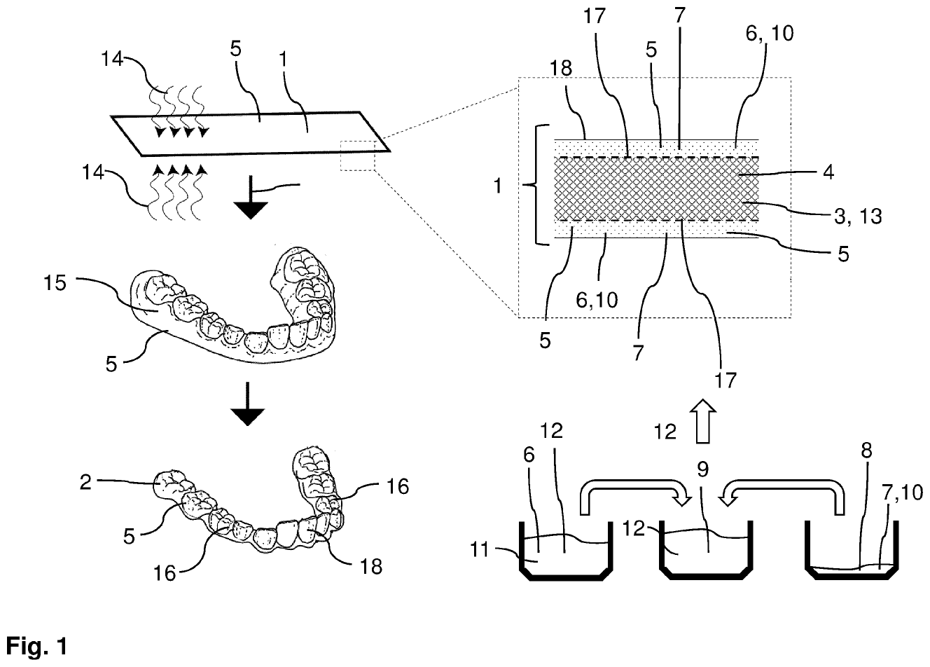

FIG. 1 provides an overview of a fabrication process according to the invention: An organic polymer 6, namely cellulose acetate butyrate (CAB), is dissolved in a solvent 12, namely acetone, to form a carrier solution 11. The carrier solution 11 is next mixed with an agent liquid 8 containing cinnamaldehyde, which is a bio-based antimicrobial agent 7, to form a carrier liquid 9, in which the agent 7 and the CAB are homogenously mixed. This carrier liquid 9 is then applied by clip coating onto a core foil 13 of a thermoplastic material 4. After evaporation of the solvent 12, a solid thermoplastic coating 5 is obtained on both sides of the core-foil 13, which thus completely covers the core 3 of the resulting functional foil 1 (c.f. the cross-section visible in FIG. 1).

The foil 1, in fact, constitutes a multi-layer structure with a core 3 made from glykol modified Polyethylenterephthalat (PETG) with a thickness of 200 μm and top and bottom functional coatings 5 which each have a thickness (after solidification) of less than 20 μm, thus resulting in a total thickness of the thermoplastic functional foil 1 of less than 250 μm.

As a next step, using a 3D-model of an oral cavity of a patient, a pre-form 15 is formed from the functional foil 1 by thermoforming, applying heat 14 from both sides to the foil 1 and thus distorting the core 3 and the coatings 5 together in one step. In other words, the coating 5 is already firmly linked to the core 3 prior to thermoforming. The underlying reason is that the acetone 12 modifies the surface of the PETG core 3 leading to a softening of the PETG surface such that the CAB molecules 6 can interlock on a nanometer scale at the interface 17 (cf. FIG. 1) with the PETG of the core 3. As a result, there is already a high adhesion of the coating 5 on the core 3 prior to thermoforming.

As the glass transition temperatures T_{g,core} of the PETG-core 3 and T_{g,coating} of the coating differ by less than 60° C., during thermoforming the adhesion of the coating 5 on the core foil 13 is further improved, as a thermal interlocking is achieved through thermal fusion of the CAB-coating 5 with the PETG.

Finally the pre-form 15 is cut such that the resulting dental appliance 2 visible on the bottom left of FIG. 1 covers both teeth and adjoining parts of the gingiva. As visible, the dental appliance 2 further features multiple cavities 16 designed for taking up single teeth and which are matching the natural positions of the teeth of the user. Thus, the user experiences no forces on his teeth when wearing the dental appliance 2 on his teeth. As the appliance 2 is very thin, highly conformable and offers a soft and highly deformable functional coating 5, which, due to the CAB used, even softens when getting into contact with saliva and offers antimicrobial protection due to the embedded cinnamaldehyde, the appliance 2 is ideally suited to be worn overnight or while smoking, even for hours. In such non-therapeutic use-cases, the user can benefit from the protection provided by the appliance 2 against cigarette smoke, as the appliance 2 is impermeable to smoke, but also from gingivitis and parodontitis, due to the antimicrobial effect that is produced when the cinnamaldehyde 7 is slowly released from the coating 5 into the oral cavity of the user.

The CAB used for the coating 5 encapsulating the core 3 as illustrated on the right of FIG. 1 is in fact a bio-based material 10, as it is produced by blending organic materials derived from fossil sources with organic material, in particular cellulose, derived from non-fossil natural sources such as plants. Therefore, the complete outer surface 18 of the appliance 2 is formed from bio-based materials 10. Any mechanical abrasion from this surface 18 will thus not be harmful for the user wearing the appliance 2 in his mouth.

Likewise, the agent 7 embedded in the coating 5 can be derived from natural sources such as essential oils extracted directly from non-fossil plants. This allows use of agents such as lime, thymol, eugenol, linalool, carvacrol, nutmeg, pimenta berry, rosemary, petitgrain, coffee, anise, to name a few. Hence the appliance 2 can deliver both flavor and antimicrobial protection and still be highly biocompatible and non-harmful to wear.

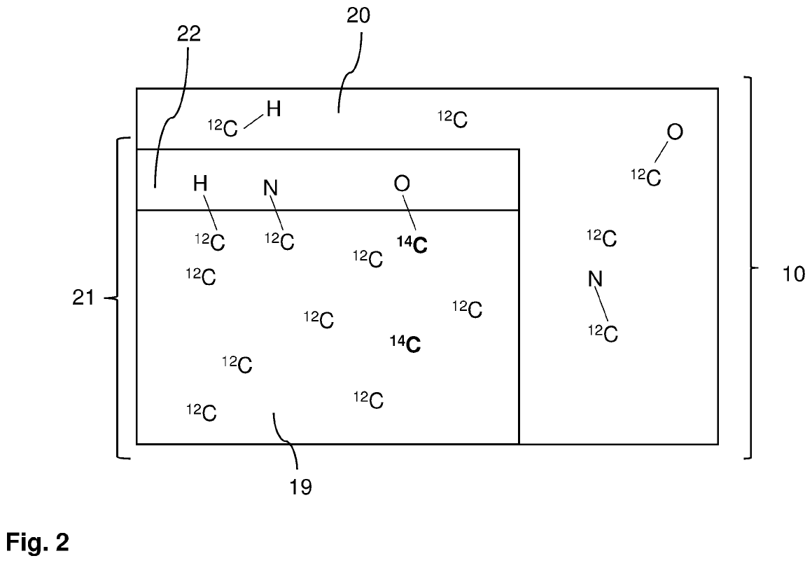

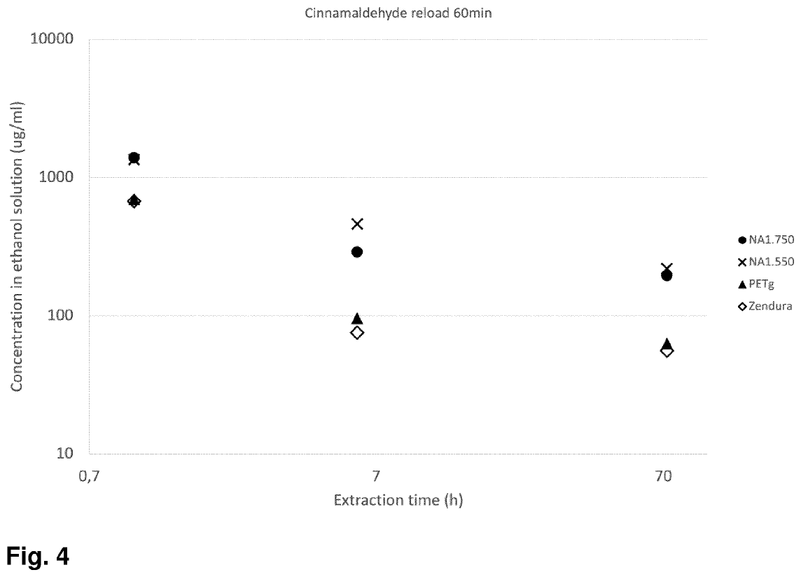

As FIG. 2 illustrates schematically, “bio-based material” as understood herein may mean that the organic polymer 6 as well as the agent 7 used for the coating 5 may contain a significant portion of non-fossil carbon content 19, for example at least 40%. This non-fossil carbon content 19 is characterized in that it contains the carbon isotope 14C in a detectable fraction (e.g. more than 10 ppq or even more than 100 ppq). There may also be a fossil carbon content 20 (cf. FIG. 4), which is based on fossil sources and containing the isotope ¹²C but no detectable fraction of ¹⁴C any more, as the ¹⁴C has been diminished by radioactive decay over thousands of years. As illustrated in FIG. 4, there can be defined a further quantity namely the so-called bio-based content 21. This fraction of the material 5 comprises the non-fossil carbon content 19 as well as all hydrogen H-, oxygen O-, and nitrogen N-atoms 22 bound to the non-fossil carbon content 19.

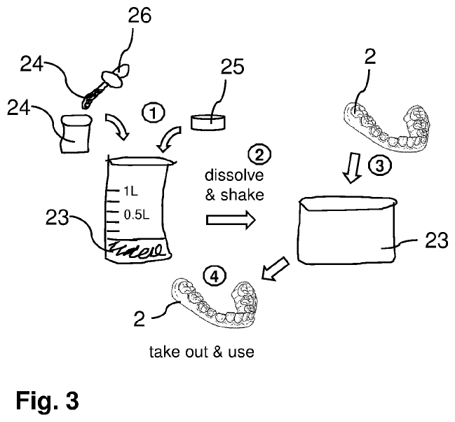

FIG. 3 illustrates the formation and use of a reload-liquid as proposed herein: In a first step, a highly concentrated liquid precursor 24 containing the agent 7 and a surfactant are first dispensed in water using a low-cost pipette 26; alternatively, a tab 25 containing the agent 7 and a surfactant may be dissolved in the water. By both ways, a reload-liquid 23 may be formed. In a second step, the reload-liquid 23 is shaken to allow thorough mixing of the liquid precursor 24 with the water and/or full dissolving of the tab 24 in the water. In a third step, the dental appliance 2 is immersed and soaked in the formed reload-liquid 23 for a duration of 15-30 minutes. Finally, in a fourth step, the dental appliance 2 is taken out of the reload-liquid 23 and is now loaded with the agent 7 and ready for use in the mouth, where the agent 7 can be released from the dental appliance 2, for example for producing a pleasant taste and/or an antimicrobial effect.

FIG. 4 displays experimental data obtained by first loading four different thermoplastic materials with an agent 7 (cinnamaldehyde) by immersing the respective thermoplastic material in a reload-liquid 23 (comprising 12 g of a surfactant and 0.56 g of the agent 7 dissolved in 100 ml of water) for a time period of 60 min, respectively. Afterwards, the materials were immersed repeatedly in fresh ethanol-solution, which was used as an extraction medium for extracting the agent 7 from the materials, and the concentration of the agent 7 in the respective ethanol-solution was measured each time. In other words, at different points in time, fresh ethanol-solutions were used for extracting the agent from the respective thermoplastic material. The graph displays the measured concentration of the agent 7 in the respective solution for each material for an extraction time of 0.7 h, 7 h, and 70 h, respectively.

As the graph shows (note the logarithmic scale on both axes!), the proposed material system (upper two symbols), comprising a cellulose-based cap layer (materials NA750 and NA550, respectively) applied onto a core of a thermoplastic material, can initially load more of the agent 7 from the same reload-liquid 23 and additionally can store the agent 7 for a much longer period of time (thus offering a lower release rate of the agent 7 over time), as compared to a poly-urethane (PU) based thermofoil (Zendura) or a pure PETG-based thermofoil (both without any coating). In bare numbers, the approach according to the invention allows for an increase in release rate by a factor of 2 . . . 6 after a reload time of only 60 minutes; the period of time during which a significant release of the agent is maintained (and thus the desired anti-microbial functionality of the dental appliance) is extended by about six times. The uptake of the cellulose-based cap layers is at least a factor of 2 . . . 4 higher, as compared to the pure core materials PETG and PU.

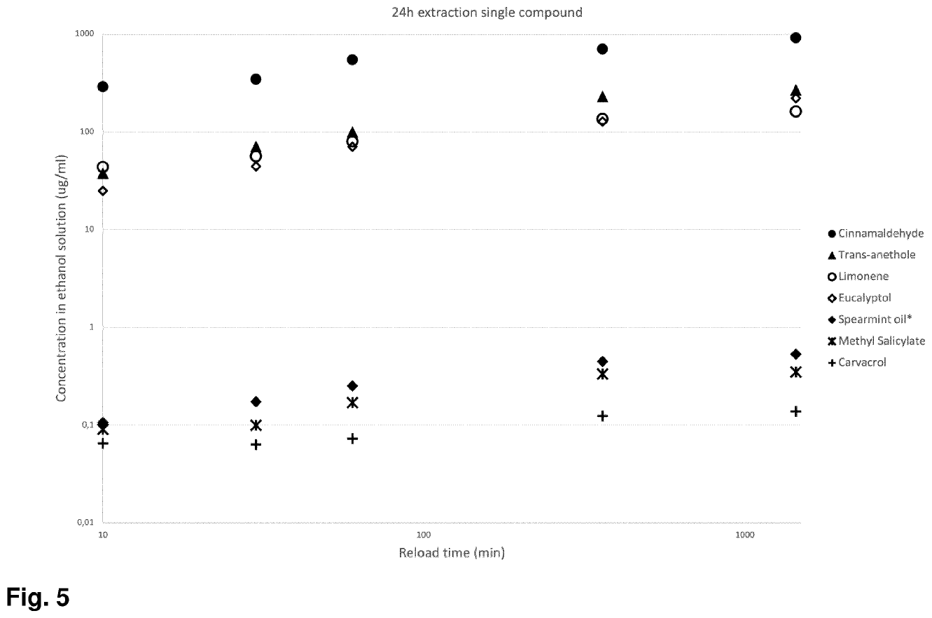

FIG. 5 displays experimental data obtained in a second experiment in which dental appliances according to the invention, each time comprising a cellulose-based cap layer, was loaded with 7 different types of agents 7 for time periods ranging from 10 to 1440 min (=24 h). The graph shows the concentration of each agent 7 in 40% ethanol-water-solution, which was used as an extraction medium over a period of 24 hours. Depending on the chemical side group (in particular its polarity) of the particular agent, the capacity to interact with the dental appliance (through storage and release) can vary by a factor of up to 10000, as can be seen by comparing the data for Carvacrol and Cinnamaldehyde. As a result, Cinnamaldehyde appears as an agent that is particularly suited for the applications described herein.

Finally, additional calorimetric experiments were performed to measure the capability of different agents of suppressing the growth of bacteria such as Streptococcus mutans and Streptococcus mitis. The different agents 7 were applied as a solution containing a single essential oil component at a concentration of 0.1% in BHI. As figure of merits, the lag phase, defined as the postponement in time of the growth of the bacteria and measured in hours, and the growth rate (increase in number of bacteria/time), measured in J/h, of the Streptococcus populations were determined as follows:

| | growth rate | lag phase (h) of S. |
|---|---|---|
| agent | (J/h) of S. mutans | mutans growth |
| Limonene | 0 | >24 |
| cinnam aldehyde | 0 | >24 |
| trans-anethole | 0.03 | >24 |
| methyl salicylate | 1.2 | 5 |
| Eucalyptol | 1.3 | 5 |

| | growth rate | lag phase (h) of S. |
|---|---|---|
| agent | (J/h) of S. mitis | mitis growth |
| methyl salicylate | 0.4 | 23 |
| trans-anethole | 0.13 | 6.4 |
| cinnam aldehyde | 0.15 | 6.2 |
| Limonene | 0.11 | 3 |
| Eucalyptol | 0.14 | 2 |

As can be seen from these data, in particular Limonene and cinnamaldehyde produce lag phases exceeding 24 hours without any measurable growth w.r.t. Streptococcus mutans. With respect to Streptococcus mitis, methyl salicylate offers the best protection due to a lag phase of 23 hours at a moderate growth rate of 0.4 J/h.

In addition, the difference between non-polar and polar agents should be noted here, since the polarity of the agent 7 will define the reload speed into the cellulose-based (e.g. CAB) coating of the dental appliance 2 as well as the extraction speed in the saliva of the patient. Furthermore the polarities of the agents 5 impact their respective interaction when combined together in one reload-liquid 23 and thus influence the reload speed of each single agent 7, depending on the ratio to each other and the ratio to the surfactant (if present in the reload-liquid 23).

As cinnamaldehyde is polar, while limonene and methyl salicylate are non-polar, cinnamaldehyde is particularly suited as an anti-microbial agent 7 to be used with a material system as proposed herein with a cellulose-based cap layer, because cinnamaldehyde offers similar anti-microbial protection but superior reload speed.

For achieving best protection against both Streptococcus mutans and Streptococcus mitis, a combination of Limonene or cinnamaldehyde and methyl salicylate or trans-anethole is proposed to be used as the agent 7 in the reload-liquid 23 described previously and thus as the antimicrobial agent 7 to be delivered by the dental appliance 2 to the user.

In summary, for improving the functionality and bio-compatibility of dental appliances 2, a novel process is proposed for making a thermoplastic functional foil 1, from which such dental appliances 2 may be obtained by thermoforming the functional foil 1. A carrier liquid 9 containing an organic and preferably bio-based polymer 6 is enriched with an agent 7 and applied onto a solid thermoplastic core foil 13. After evaporating of a solvent 12 contained in the carrier liquid 9, a uniform and highly homogenous functional coating 5 is obtained on the core foil 13. After thermoforming of the foil 1, the dental appliance 2 thus features an outer protective coating 5 offering enhanced functionality (c.f. FIG. 1). Moreover, it is possible to reload the agent 7 into the coating 5 of the appliance 2 using a reload-liquid.

### LIST OF REFERENCE NUMERALS

1 thermoplastic functional foil
2 dental appliance
3 core
4 thermoplastic material
5 thermoplastic coating
6 organic polymer
7 agent
8 agent liquid
9 carrier liquid
10 bio-based material
11 carrier solution
12 solvent
13 core foil
14 heat applied
15 pre-form
16 cavities (for taking up single teeth)
17 interface
18 outer surface
19 non-fossil carbon content (containing ¹⁴C)
20 carbon content based on fossil sources (containing no detectable
fraction of ¹⁴C any more)
21 bio based content (i.e. non-fossil carbon content plus hydrogen,
nitrogen, and oxygen bound to this content)
22 hydrogen, nitrogen, and oxygen bound to non-fossil carbon
23 reload-liquid
24 liquid precursor
25 tablet
26 pipette

1. A process for making a thermoplastic functional foil (1) comprising a core (3) of thermoplastic material (4) and a thermoplastic coating (5) at least partially covering the core (3), the process comprises the following steps:
providing a carrier liquid (9) comprising an organic polymer (6);
adding an agent (7), releasable from the thermoplastic coating (5) in aqueous environment, to the carrier liquid (9) prior to forming the thermoplastic coating (5); and
applying the carrier liquid (9) as the thermoplastic coating (5) onto the core (3).

2. The process according to claim 1,
wherein the agent (7) is derived from a renewable biological resource comprising a material of non-fossil origin produced by a living organism.

3. The process according to claim 1,
wherein the agent (7) comprises at least one of: an essential oil, an extract of an essential oil, cinnamaldehyde, lime, thymol, eugenol, linalool, carvacrol, nutmeg, pimenta berry, rosemary, petitgrain, coffee, or anise.

4. The process according to claim 1,
wherein at least one of a) the organic polymer (6) is derived from a renewable biological resource,
b) the organic polymer (6) is a cellulose-based material, or
the organic polymer (6) is cellulose acetate butyrate (CAB).

5. The process according to claim 1,
wherein the coating (5) provides antimicrobial protection.

6. The process according to claim 1,
wherein a melting temperature T_{m,core} of the core (3) and a melting temperature T_{m,coating} of the coating (5) differ by at least 20° C.

7. The process according to claim 1,
wherein the core (3) is made from Polyethylenterephthalat (PET).

8. The process according to claim 1,
wherein the agent (7) comprises a combination of essential oils as follows:
Limonene or cinnamaldehyde
and
methyl salicylate or trans-anethole;

9. The process according to claim 1,
wherein a glass transition temperature T_{g,coating} of the coating (5) is from 80-165° C.,
and at least one of a) a decomposition temperature T_{d,agent} of the agent (7) is at least 10° C. above a melting temperature T_{m,coating} of the coating (5),
b) a corresponding melting range of the coating (5) is from 120-200° C.,
or
c) the organic polymer (6) used for the coating (5) has a number average molecular weight of more than 12.000 g.

10. The process according to claim 1,
wherein the carrier liquid (9) is a carrier solution (11), and
the core (3) is a core foil (13).

11. The process according to claim 1, further comprising,
forming the coating (5) by a physical coating process including at least one of spin coating, blade-based coating, spray coating, or by roll-to-roll coating.

12. The process according to claim 1,
wherein a glass transition temperature T_{g,core} of the core (3) and a glass transition temperature T_{g,coating} of the coating (5) differ by less than 80° C.

13. The process according to claim 1,
wherein the carrier liquid (9) is a carrier solution (11) obtained by dissolving the organic polymer (6) in a solvent (12), and at least one of a) the solvent (12) modifies a surface of the core (3) such that the core (3) and the coating (5) interlock on a nanometer scale, resulting in improved adhesion of the coating (5) on the core (3), or
b) the solvent (12) comprises at least one of acetone, methyl acetate, ethyl acetate, methylethyl ketone, isopropyl acetate, butyl acetate, ethyl lactate, cyclohexane, diacetone alcohol, butyl lactate or suitable mixtures thereof.

14. The process according to claim 1,
wherein the agent (7) is a liquid or
the agent (7) is added to the carrier liquid (9) by dissolving or emulsifying the agent (7) in an aqueous or oil-based solution and mixing the solution with the carrier liquid (9).

15. The process according to claim 1,
wherein a ratio between the agent (7) and the organic polymer (6) is between 0.01/99.99 and 30/70 by weight.

16. The process according to claim 1, further comprising
forming a dental appliance (2) from the thermoplastic functional foil (1) by thermoforming the foil (1) after application of the coating (5) to the core (3), after thermoforming, the organic polymer (6) forms a conformal functional coating (5), and
the agent (7) embedded in the coating (5) provides antimicrobial or regenerative functionality for teeth and gingiva or a flavor to the dental appliance (2).

17. A dental appliance (2), comprising:
the thermoplastic functional foil (1) fabricated with the process according to claim 1, wherein the dental appliance (2) consists entirely of the foil (1) or
the dental appliance is 3D-printed from a liquid precursor comprising an organic polymer and loaded with a releasable agent (7).

18. A dental appliance (2), comprising:
a core (3) of thermoplastic material (4),
a thermoplastic coating (5) at least partially covering the core (3), and
an agent (7) embedded in the coating (5) and releasable from the coating (5) in aqueous environment.

19. A method for non-therapeutic protecting of at least one of teeth or gingiva from gingivitis and/or parodontitis, the method comprising:
covering both teeth and parts of the gingiva adjoining to the teeth with the dental appliance according to claim 18,
wherein the dental appliance (2) provides antimicrobial protection through the agent comprising a releasable antimicrobial agent (7).

20. A method for loading or re-loading a dental appliance (2) with a releasable agent (7), the method comprising:
providing a reload-liquid that contains the agent (7), and
immersing the dental appliance (2) in the reload-liquid to load the agent (7) into the dental appliance (2).

21. The method according to claim 20, wherein the reload-liquid (23) is
an aqueous solution containing a solvent
or
an oil-based liquid.

22. The method according to claim 21, wherein the reload-liquid (23) is obtained by dissolving a solid tablet (25) containing the agent (7) in a liquid.

23. The method according to claim 22, wherein at least one of the reload-liquid (23) or the tablet (25) comprises a surfactant for homogenously distributing the agent (7) within the reload-liquid (23).

24. The method according to claim 23,
wherein at least one of a) a concentration of the surfactant cₛ in the reload-liquid (23) is cₛ>1.1 g/l, or
b) a ratio of a concentration cₛ of the surfactant and a concentration cₐ of the agent (7) in the reload-liquid (23) is cₛ/cₐ<100.

25. The method according to claim 24, wherein the agent (7) is at least one of
an antimicrobial agent (7),
derived from a renewable biological resource,
a combination of the following essential oils:
Limonene or cinnamaldehyde
and
methyl salicylate or trans-anethole.

26. A method for initially loading a dental appliance (2) with a releasable agent (7), the method comprising:
embedding the releasable agent (7) in a thermoplastic coating (5) of the dental appliance (2) by immersing the dental appliance (2) in a reload-liquid (23) that contains the releasable agent (7), thereby loading the releasable agent (7) into the thermoplastic coating (5); or
embedding the releasable agent (7) in a 3d-printed body of the dental appliance (2) by immersing the dental appliance (2) in a reload-liquid (23) that contains the releasable agent (7), thereby loading the releasable agent (7) into the 3d-printed body.

27. The dental appliance of claim 17, wherein the releasable agent (7) is at least one of:
a) releasable from the dental appliance (2) during intra-oral use, or
b) reloadable into a cap layer of the dental appliance (2) using a reload-liquid (23).
